(19) United States
(12) Patent Application Publication
(10) Pub. No.: US 2026/0359916 A1
(43) Pub. Date: Nov. 12, 2026

(54) System and Method for AI-Based Real-Time Interaction, HTML Parsing, and PDF Generation in a Healthcare Web Application for Enhancing the Doctor-Patient Relationship using Personalized Patient Education

(71) Applicant: JASON JOHN PIROZZOLO, KEY WEST, FL (US)

(72) Inventor: JASON JOHN PIROZZOLO, KEY WEST, FL (US)

(21) Appl. No.: 19/369,257

(22) Filed: Oct. 26, 2025

(51) Int. Cl. G06Q 50/20; G16H 10/60

(52) U.S. Cl. CPC G06Q 50/205; G16H 10/60

(57) ABSTRACT

A web-based system generates real-time, personalized patient education during a clinical encounter. A server receives clinical context from a clinician device, parses HyperText Markup Language (HTML) educational content, and applies a natural language processing module to adjust readability, tone, and language. A personalization engine inserts patient-specific instructions. A PDF generator produces a formatted file that is delivered to a patient user interface and optionally logged in a clinician dashboard, with storage in a repository and optional link sharing via a messaging gateway. The system improves doctor-patient communication by creating understandable, printable instructions tailored to each patient's condition.

System and Method for AI-Based Real-Time Interaction, HTML Parsing, and PDF Generation in a Healthcare Web Application for Enhancing the Doctor-Patient Relationship using Personalized Patient Education

### BACKGROUND

Patients often leave medical visits unsure about diagnosis, instructions, or next steps. Generic handouts do not match each patient's condition, reading level, or language. Clinicians have limited time to create personalized education during the visit. There is a need for a system that generates clear, patient-specific materials in real time.

### SUMMARY

The invention is a web application that produces personalized patient education during a clinical encounter.
A server (102) receives clinical context from a clinician on a client device (104).
An HTML parser (108) extracts relevant sections from educational content.
An NLP module (106) adapts tone and readability to patient attributes.
A personalization engine (112) inserts patient-specific items (e.g., meds, activities).
A PDF generator (110) creates a formatted file.

The result is delivered to a patient UI (114) and optionally logged in a clinician dashboard (116), with storage in a database or repository (118) and optional distribution through a messaging gateway (120).

### BRIEF DESCRIPTION OF THE DRAWINGS

FIG. 1 shows a system environment (100) including server (102), client device (104), and storage (118).

FIG. 2 shows modules: NLP (106), HTML parser (108), PDF generator (110), personalization engine (112), patient UI (114), clinician dashboard (116), and messaging gateway (120).

FIG. 3 shows data flow among these modules.

FIG. 4 shows a sequence for real-time interaction during a visit.

FIG. 5 shows a flowchart of the method to generate and deliver a PDF.

FIG. 6 shows an example patient interface (114).

FIG. 7 shows an example clinician dashboard (116).

FIG. 8 shows an example PDF layout.

### DETAILED DESCRIPTION

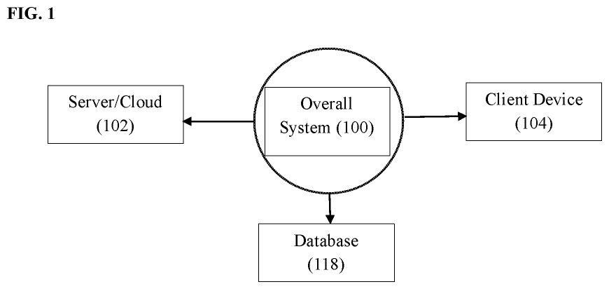

Referring to FIG. 1, a system environment (100) includes a networked server (102) that communicates with one or more client devices (104) used by clinicians and patients. The server (102) accesses storage such as a database or repository (118) and can send links or files via a messaging gateway (120).

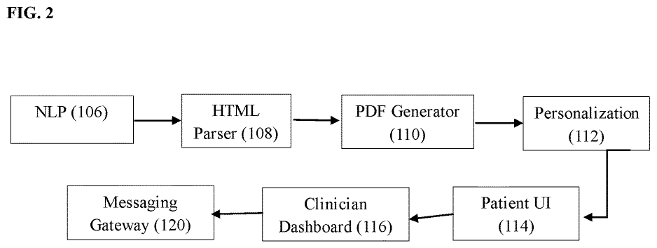

As shown in FIG. 2, the server (102) hosts software components:
NLP module (106): transforms text for clarity, target reading level, tone, and language.
HTML parser (108): parses HTML content (e.g., educational articles) to extract sections relevant to a patient's diagnosis, symptoms, or plan.
PDF generator (110): creates a printable PDF with headings, lists, and optional logo or header.
Personalization engine (112): merges clinician inputs (e.g., job demands, hobbies, dosing, cautions) and patient attributes (e.g., age, language).
Patient UI (114): lets a patient view and download the generated content.
Clinician dashboard (116): lets a clinician enter context, preview the education, approve, and send.
Messaging gateway (120): provides delivery or link sharing (e.g., secure link or cloud drive link).
Database/Repository (118): stores generated artifacts, metadata, or links.

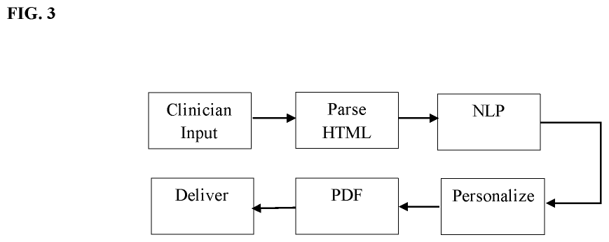

Data flow (FIG. 3). During a visit, the clinician device (104) sends clinical context (e.g., sex, age, job, hobbies, diagnosis) to the server (102). The personalization engine (112) retrieves or receives educational HTML. The HTML parser (108) extracts relevant headings and paragraphs. The NLP module (106) simplifies the text, adjusts tone, and, if needed, translates it. The PDF generator (110) formats the final content. The server (102) delivers the result to the patient UI (114) and records entries in storage (118). The messaging gateway (120) may return a shareable link.

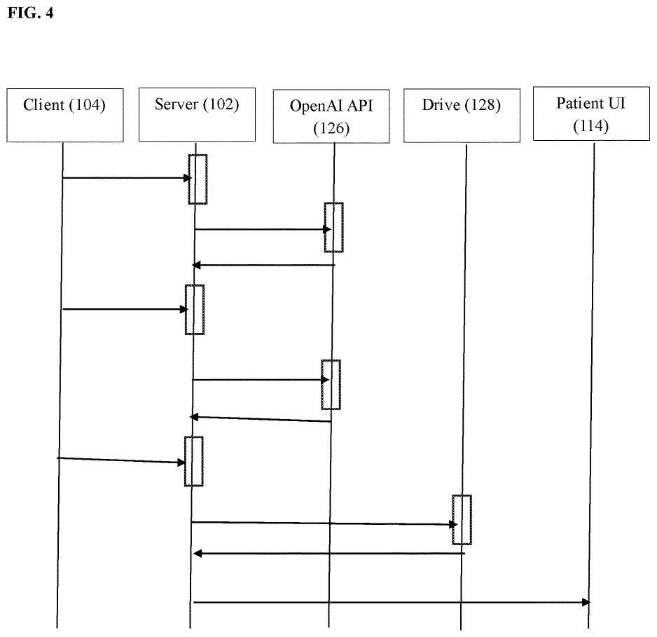

Real-time sequence (FIG. 4). The clinician enters context→the system parses and rewrites content→a preview appears on the dashboard (116)→the clinician approves→the patient sees it on the patient UI (114) and can download a PDF.

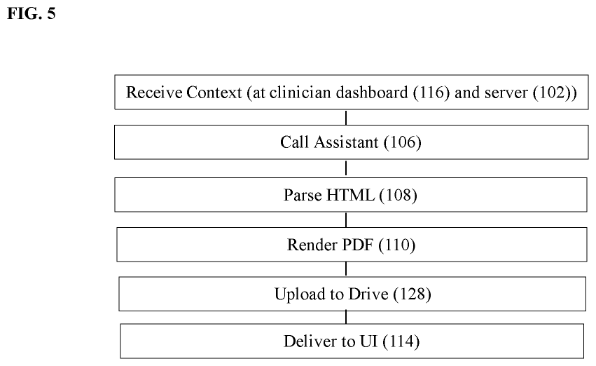

Method flow (FIG. 5). The method includes:
(1) receiving clinical context;
(2) retrieving HTML educational content;
(3) parsing relevant sections;
(4) transforming with NLP to target readability/tone and language;
(5) personalizing with patient-specific instructions;
(6) generating a formatted PDF;
(7) delivering to the patient UI and optionally logging/storing.

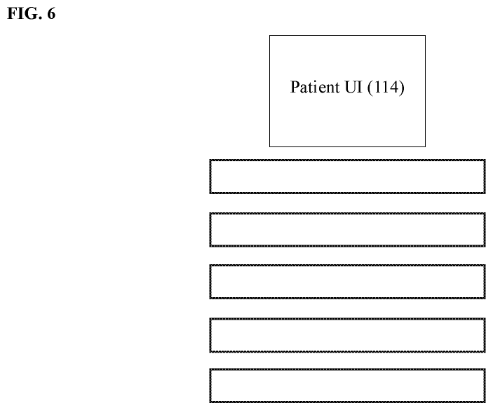

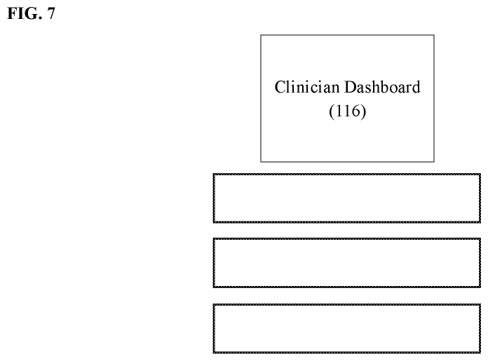

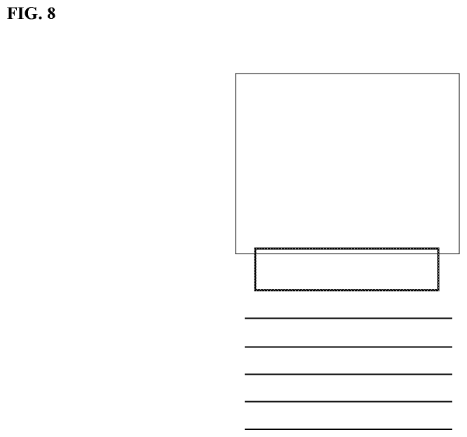

Interfaces (FIG. 6-8). The patient UI (114) shows a readable summary and a “Download PDF” button. The clinician dashboard (116) shows form fields for context and an approval button. The PDF (FIG. 8) includes a header, condition overview, step-by-step instructions, lifestyle guidance, and follow-up details.

Implementation notes. In one embodiment, the server (102) uses a web framework to expose endpoints for: generating patient-specific text based on clinician-provided inputs, translating a response into a target language, parsing provided HTML content, producing a PDF via a PDF library, storing the PDF or a link in the repository (118), and returning metadata (e.g., a link) via HTTP response headers. The NLP module (106) may call an AI service for generation and translation. The messaging gateway (120) may be implemented by a cloud file API that returns a viewable link accessible to the patient after approval.

Privacy and HIPAA Safeguards (non-limiting). In one embodiment, the server (102) is configured so that no protected health information (PHI) is sent to third-party services. Requests from the server to any external AI service API (126) transmit only de-identified content; direct identifiers (e.g., name, full street address, medical record number, full dates) are removed or tokenized prior to transmission. When PHI handling is enabled within the covered entity, PHI remains under the covered entity's control: all PHI is encrypted in transit and at rest, access is restricted by role-based controls, and accesses/changes are recorded via audit logging (124) under a minimum-necessary policy. The messaging gateway (120) issues authenticated, time-limited links for patient access through the patient UI (114), and the system avoids sending unencrypted PHI via public channels. The system can require a Business Associate Agreement (BAA) with any vendor before PHI is transmitted; absent a BAA, outbound requests are automatically de-identified. These safeguards are provided to support HIPAA compliance without limiting the scope of the claims; other equivalent privacy controls may be used.

### EXAMPLES

### Example 1 (Prophetic)

A clinician opens the dashboard (116) and enters: sex=female, age=45, job=teacher, hobbies=hiking, diagnosis=“asthma follow-up.” The system retrieves asthma management content in HTML, the parser (108) extracts the “daily control” and “rescue steps” sections, and the NLP (106) rewrites them to a sixth-grade reading level with friendly tone. The personalization engine (112) inserts the prescribed inhaler name and dosing as provided by the clinician. The PDF generator (110) creates “Dr_Jasons_10_22_2025_Asthma_Teacher_Hiking. pdf”. The server (102) delivers a preview to the dashboard (116) and a download to the patient UI (114). A link to the file is stored in the repository (118) and may be shared via the messaging gateway (120).

### INDUSTRIAL APPLICABILITY

The system is useful in hospitals, clinics, and telemedicine platforms. It improves patient understanding, supports in-visit education, and reduces clinician workload by automating personalized materials.

1. A computer-implemented method for generating personalized patient education in real time, the method comprising:
(a) receiving, at a server from a clinician device, clinical context for a patient during an encounter;
(b) retrieving, by the server, educational content in HyperText Markup Language (HTML) responsive to the clinical context;
(c) parsing, by an HTML parser, the educational content to extract sections relevant to the clinical context;
(d) transforming, by a natural language processing (NLP) model, the extracted sections to a target readability and tone based on patient attributes;
(e) personalizing, by a personalization engine, the transformed sections with patient-specific instructions supplied by the clinician;
(f) generating, by a PDF generator, a formatted portable document file (PDF) comprising the personalized content; and
(g) delivering, by the server, the PDF and a corresponding preview to a patient user interface for display or download during the encounter.

2. The method of claim 1, further comprising retrieving structured patient data from an electronic health record (EHR) system and using the data to populate medication names, dosages, appointments, or contact details.

3. The method of claim 1, further comprising applying medical-safety filters that detect contraindications or missing instructions and prompting the clinician to confirm or edit flagged portions before delivery.

4. The method of claim 1, wherein generating the PDF comprises selecting a layout template from a plurality of predefined templates based on the clinical context or patient attributes.

5. The method of claim 1, further comprising storing an audit log entry including a timestamp, clinician identifier, patient identifier, and a version hash of the delivered PDF.

6. The method of claim 1, further comprising caching intermediate NLP results keyed to the clinical context to reduce computation on subsequent requests during the same encounter.

7. The method of claim 1, wherein delivering the PDF meets a latency target under a predetermined threshold to enable in-visit review.

8. The method of claim 1, wherein generating the PDF further inserts a standardized medical disclaimer and contact instructions on a final page.

9. The method of claim 1, further comprising automatically translating the personalized content into a patient language preference while preserving medical terminology with glossary notes.

10. The method of claim 1, further comprising enforcing role-based access control that requires clinician authentication to approve delivery and patient authentication to view or download.

11. The method of claim 1, further comprising capturing analytics including page views, link clicks, and completion of patient-education acknowledgments, and storing aggregated metrics in the database.
